(19) United States
(12) Patent Application Publication
(10) Pub. No.: US 2026/0315424 A1
(43) Pub. Date: Oct. 15, 2026

(54) OXYLIPINS IN THE TREATMENT OF SEBORRHEA

(71) Applicant: PIERRE FABRE DERMO-COSMETIQUE, Lavaur (FR)

(72) Inventors: Mathieu LETI, Castres (FR); Christophe DUNOUAU, Castres (FR); Lucile GARIDOU, Castres (FR); Céline MIAS, Castres (FR); Nicolas STEWARD, Castres (FR)

(73) Assignee: PIERRE FABRE DERMO-COSMETIQUE, Lavaur (FR)

(21) Appl. No.: 19/481,064

(22) PCT Filed: Apr. 26, 2024
(86) PCT No.: PCT/EP2024/061632

§ 371 Date: Nov. 3, 2025

(30) Foreign Application Priority Data
May 4, 2023 (FR) FR2304484

(51) Int. Cl. A61K 31/201; A61K 31/557; A61K 36/03; A61K 36/48; A61K 36/73; A61P 17/08

(52) U.S. Cl. CPC A61K 31/201; A61K 31/557; A61K 36/03; A61K 36/48; A61K 36/73; A61P 17/08; A61K 2236/333; A61K 2236/39

(57) ABSTRACT

The present invention relates to oxylipins for use in the treatment of seborrhea, the oxylipins more particularly being present in a preferably oxylipin-enriched oleaginous plant extract. The present invention also relates to a dermo-cosmetic or dermatological composition comprising at least one oxylipin, preferably derived from an oleaginous plant, with at least one dermo-cosmetically or dermatologically acceptable excipient, for use in the treatment of seborrhea.

### TECHNICAL FIELD OF THE INVENTION

The present invention concerns oxylipins, and in particular plant extracts containing them, as well as cosmetic or dermatological compositions comprising them, for their use in seboregulation, and in particular for the treatment of seborrhea and/or acne, preferably seborrhea.

### STATE OF THE ART

Living organisms are formed by one or more cells, prokaryotes or eukaryotes, whose survival, defense and proliferation functions rely on the metabolism of three major chemical families: carbohydrates, proteins and lipids.

Lipids are natural substances; their roles are fundamental. They are major constituents of cellular structures, such as membrane phospho- and glycolipids. They are also reserve substances, as a source of energy for the organism. They are involved in cellular defense or signaling mechanisms and constitute coating elements such as waxes or cutins (Cuvelier et al., 2004).

Lipids are hydrophobic and sometimes amphiphilic substances, soluble in apolar or moderately polar organic solvents.

Simple lipids, esters of fatty acids and an alcohol which can be glycerol (constitutive of triglycerides) or an aliphatic alcohol of high molecular mass (constitutive of ceramides), can be distinguished from complex lipids such as phospholipids or glycolipids.

Fatty acids are the elementary building blocks of lipids. They can have two additive origins in an organism: synthesis by the organism and diet.

Fatty acids are aliphatic monocarboxylic acids of variable length. The most common natural fatty acids have between 4 and 28 carbon atoms. The length of their carbon chain and the number and type of their unsaturation(s) are also variable. Saturated, monounsaturated (a single double bond) and polyunsaturated (several double bonds) fatty acids are distinguished. Unsaturated fatty acids can contain 1 to 6 double bonds in living organisms. For the same chemical formula, there can be a large number of isomers, depending on the position of the double bond but also on its cis or trans conformation.

In the plant kingdom, fatty acid can be oxidized in the ketone form (e.g. licanic acid), hydroxylated form (e.g. ricinoleic acid) or epoxy form (e.g. vernolic acid).

In general, triglycerides are heterogeneous and a vegetable oil is a complex mixture of triesters.

In the plant world, triacylglycerols are usually stored as oily inclusions (oleosomes from the endoplasmic reticulum) which sometimes coalesce into large clusters in the cells of reserve tissues. This is particularly true for seeds, in which they can account for more than 50% of the dry mass. Exceptionally, the seed may accumulate esters of fatty acids and long chain aliphatic alcohols (e.g. jojoba) rather than triglycerides. Although this is less common, there are fruits that concentrate triglycerides in their pericarp (olive, avocado, bay laurel, etc.). Omega-3 and omega-6 fatty acids, essential in humans, such as arachidonic acid (C20:4 n-6), eicosapentaenoic acid (C20:5 n-3) and docosahexaenoic acid (C22:6 n-3) cannot be synthesized by the vertebrate enzyme arsenal without the essential supply of linoleic acid and alpha-linolenic acid. These nutrients are therefore called vital and must necessarily be provided by food. These polyunsaturated fatty acids are predominantly present in food as triacylglycerols, sterol esters, phospholipids, esterified or not or in free form (Liu et al., 2015). Major plant sources of linoleic acid include soybean, sunflower, safflower, evening primrose, and rice bran oils.

Major plant sources of alpha-linolenic acid include rapeseed, flaxseed, camelina, chia, canola, soy, perilla or walnut oils.

Several oleaginous macroalgae and microalgae are also sources of polyunsaturated fatty acids (Van Ginneken et al., 2011).

Polyunsaturated fatty acids are the substrate for many enzymatic or non-enzymatic reactions that produce oxygenated metabolites with various functions called oxylipins. Oxylipins comprise oxidized polyunsaturated fatty acids and derivatives thereof. They are found in mammals, flower plants (angiosperms), mosses, algae, bacteria and fungi. Oxylipins include signaling molecules, ligands to transcription factors, or precursors of lipid mediator synthesis (Calder, 2012). For example, the class of eicosanoids, including leukotrienes and prostanoids, is a large family of oxidation derivatives of polyunsaturated fatty acids containing 20 carbon atoms. Docosanoids are enzymatic oxidation products of polyunsaturated fatty acids with 22 carbon atoms such as docosahexaenoic acid. Oxidation products of polyunsaturated fatty acids with 18 carbon atoms such as linoleic acid or gamma-linolenic acid belong to the octadecanoid class. Octadecanoids derived from linoleic acid include 9-hydroxyoctadecadienoic acid (9-HODE) and 13-hydroxyoctadecadienoic acid (13-HODE). These two molecules are described as an in vivo oxidation marker (Lagarde, 2011). Hydroperoxy, epoxy, or ketone forms derived from linoleic acid are also documented (Richardson et al., 2017). The best-known oxygenated metabolite of gamma-linolenic acid is the one leading to jasmonic acid, a cyclic derivative with well-described phytopathogen resistance activities (Blée, 2002).

Oxidation of polyunsaturated fatty acids can be carried out non-enzymatically as well as enzymatically (Oenel et al., 2017). For the latter, the position of the unsaturations on the polyunsaturated fatty acid as well as the enzymatic environment will define the type of oxylipins formed. The main pathways are those of cyclooxygenases, lipoxygenases and cytochrome P-450 (Andreou et al., 2009). While the enzymatic pathway seems to be the main pathway for the formation of oxygenated fatty acids from polyunsaturated fatty acids in free form, some studies show that the oxidation of polyunsaturated fatty acids, especially in their esterified form (especially triglycerides), can be done via a non-enzymatic pathway. This can occur in the oleaginous plant matrix by the action of free radicals whose formation can be favored by the storage conditions (temperature, duration, etc.). These oxidized fatty acids can then be released after deesterification, especially by the action of lipases (Oenel et al., 2017). Oxylipins are present in polyunsaturated vegetable oils or oleaginous polyunsaturated fatty acid plant matrices of plants or algae. Algae can be sources of polyunsaturated fatty acids and contain enzymes that form oxylipins (Richardson et al., 2017). Their synthesis can also be promoted in low oleaginous tissues under certain conditions, such as tobacco leaves exposed to cryptogein (Rusterucci et al., 1999). Finally, patent application FR2789085 describes a method for obtaining an oil enriched in hydroxyoctadecadienoic fatty acids and especially in 9-hydroxyoctadecadienoic acid from an oily mixture containing linoleic acid. Hydroxyoctadecadienoic fatty acids are obtained by controlled oxidation of linoleic acid and/or alpha and/or gamma linolenic acid or their esters in the presence of an oxidation catalyst such as iron or copper halide.

The sebaceous gland is an exocrine gland present on the entire skin, except the palms of the hands and feet. It is found more particularly in the seborrheic regions of the face, in particular in the T zone, i.e., the middle zone of the face that forms a T, the back and the torso. It is usually associated with a hair follicle. This is why we talk about the pilosebaceous unit. The sebaceous gland is responsible for the synthesis and secretion of sebum by the sebocytes, the epithelial cells that compose it.

Sebum is a complex of lipids synthesized under hormonal stimulation. It is an essential component of the hydrolipidic film that protects the skin from external aggressions and dehydration by playing a major barrier role. Associated with this barrier function, sebum also has antibacterial properties, via oleic and palmitoleic acids and an antioxidant power provided by vitamin E. Thus, sebum helps maintain the integrity of the skin barrier.

Seborrhea corresponds to an excessive secretion of sebum by the sebaceous glands. It causes aesthetic issues. The skin has a shiny appearance; the complexion is dull and the pilosebaceous orifices are dilated. In addition, make-up does not exhibit good hold on this type of so-called oily skin. For seborrhea of the scalp, the hair has a greasy and dull appearance and is difficult to style. When seborrhea is intense, it is said to be oily and fluent, and it can be associated with a rancid smell.

Acne is a chronic inflammatory skin disease of this pilosebaceous unit leading to the formation of retentional lesions, that is to say open and/or closed comedones. It is important not to trivialize this condition and to treat it correctly because it can lead to disabling psychosocial consequences, especially due to the formation of scars.

During acne, under the influence of various endogenous factors, mainly hormonal, but also external factors such as pollution, poor diet, stress, etc., there is a deregulation of the sebaceous gland affecting the quantity and quality of sebum. Indeed, the skin of acne patients is characterized by an increase in the secretion of sebum, called hyperseborrhea, as well as a modification of its overall composition called dysseborrhea. The lipid ratios present in sebum from subjects with acne differ from sebum from healthy skin, with a significant increase in free fatty acid levels, squalene and its oxidation, and waxes.

This modified hyperseborrhea, i.e., more sebum with a thicker consistency, will participate, with dyskeratosis, in the obstruction of the follicular canal. In addition, it will constitute a culture medium favorable to the development of pro-inflammatory acne bacteria, such as Cutibacterium acnes, the most described to date. All of these elements will therefore lead to the appearance of the first acne lesions, open and closed comedones, which can evolve toward more severe inflammatory lesions, papules, pustules and nodules.

Thus, there is always a need to provide effective solutions to treat seborrhea, and therefore also acne, and to resolve the aesthetic issues associated with seborrhea.

### SUMMARY OF THE INVENTION

The object of the present invention is therefore to meet these needs. In fact, the inventors have surprisingly demonstrated that extracts enriched in oxylipins derived from different plants have the capacity to reduce the production of sebum. Such an anti-seborrheic activity is particularly advantageous in patients with acne skin.

The present invention also relates to at least one oxylipin, and more particularly a mixture of oxylipins such as a plant extract containing or enriched in oxylipins, for its use in the treatment of seborrhea and/or acne, preferably seborrhea.

The invention also concerns the use of at least one oxylipin, and more particularly a mixture of oxylipins such as a plant extract containing or enriched in oxylipins, for the preparation of a dermo-cosmetic or dermatological composition intended for the treatment of seborrhea and/or acne, preferably seborrhea.

The invention also concerns the use of at least one oxylipin, and more particularly a mixture of oxylipins such as a plant extract containing or enriched in oxylipins, for the treatment of seborrhea and/or acne, preferably seborrhea.

The invention also concerns a method for treating seborrhea and/or acne, preferably seborrhea, comprising administering to a person in need thereof an effective quantity of at least one oxylipin, and more particularly a mixture of oxylipins such as a plant extract containing or enriched in oxylipins.

The present invention also relates to a dermo-cosmetic or dermatological composition comprising at least one oxylipin, and more particularly a mixture of oxylipins such as a plant extract containing or enriched in oxylipins, with at least one dermo-cosmetically or dermatologically acceptable excipient, for its use in the treatment of seborrhea and/or acne, preferably seborrhea.

The invention also concerns the use of a dermo-cosmetic or dermatological composition comprising at least one oxylipin, and more particularly a mixture of oxylipins such as a plant extract containing or enriched in oxylipins, with at least one dermo-cosmetically or dermatologically acceptable excipient, for the preparation of a medicament intended for the treatment of seborrhea and/or acne, preferably seborrhea.

The invention also concerns a method for treating seborrhea and/or acne, preferably seborrhea, comprising administering to a person in need thereof an effective quantity of a dermo-cosmetic or dermatological composition comprising at least one oxylipin, and more particularly a mixture of oxylipins such as a plant extract containing or enriched in oxylipins, with at least one dermo-cosmetically or dermatologically acceptable excipient.

In the context of the present invention, the oxylipin may preferably be a mixture of oxylipins. The oxylipin will more particularly be derived from a plant and more particularly from an oleaginous plant material and may be present in a plant extract, and more particularly a plant extract enriched in oxylipins. The plant extract may more particularly be an oleaginous plant extract, preferably enriched in oxylipins.

### Definitions

In the present invention, “approximately” means that the value concerned may be 10%, especially 5%, in particular 2%, more particularly 1% lower or higher than the value indicated. In the present invention, “extract” or “plant extract” means the product obtained after extraction of the plant or of a part of the plant, such as a vegetable oil derived from the plant, with a solvent called extraction solvent, which may then optionally be in a concentrated or dry form after partial or total evaporation of the extraction solvent. In particular, it may be a dry extract. Extraction can also be done by mechanical processes such as pressure.

In the present invention, “dry extract” means an extract devoid of extraction solvent or containing it solely in the form of insignificant traces. Such a dry extract thus contains only material derived from the plant raw material. It may also contain insignificant traces of extraction solvent.

In the present invention, “oleaginous plant material” means an oleaginous plant or part of a plant, i.e., a plant rich in fat. Examples include oleaginous seeds or fruits (e.g. apple seeds, soybeans, linseeds, safflower seeds, cumin seeds, camelina seeds, canola seeds, perilla seeds, chia seeds, rapeseed seeds, hazelnuts or a mixture thereof), gametophytes (e.g. brown algae, in particular Saccharina latissima) or a mixture thereof. It may also include a vegetable oil (e.g. apple seed oil, soybean oil, linseed oil, safflower oil, cumin oil, rice bran oil, camelina oil, canola oil, perilla oil, chia oil, rapeseed oil, hazelnut oil or a mixture thereof).

In the present invention, “oleaginous plant extract” means an extract as defined above obtained from an oleaginous plant material.

In the present invention, “fatty acid” means a carboxylic acid R1CO₂H in which the chain R1 is a linear or branched hydrocarbon chain, saturated or comprising one or more, especially 1, 2, 3, 4, 5 or 6, C═C double bonds, the carboxylic acid comprising from 10 to 28, preferably from 14 to 24, in particular from 16 to 22, carbon atoms (including the carbon atom of the carboxylic acid function).

In the present invention, “polyunsaturated fatty acid” means a fatty acid as defined above in which the hydrocarbon chain comprises one or more, especially 1, 2, 3, 4, 5 or 6, C═C double bonds.

In the present invention, “free fatty acid” means a fatty acid not bound to other molecules (for example to glycerol or derivatives thereof to give glycerides or to an alcohol to give a fatty ester).

In the present invention, “oxylipin” means an oxygenated molecule derived from the enzymatic or non-enzymatic oxidation of polyunsaturated fatty acids. The oxidation of polyunsaturated fatty acid makes it possible in particular to introduce one or more, especially 1, 2 or 3, oxygenated groups especially chosen from hydroxy (—OH), hydroperoxy (—OOH), keto (═O; the oxygen being bound by a double bond to a carbon atom) and epoxy (—O—; the oxygen being bound to two adjacent carbon atoms) groups, preferably 1, 2 or 3 hydroxy and/or keto groups, in particular with displacement of one or more, especially 1, 2 or 3, C═C double bonds so as to create a conjugation with another double bond and/or replacement of a C═C unit by a C(GO1)-C(GO2) unit where GO1 and GO2 each represent, independently of one another, an oxygenated group as defined above. Oxylipins can be in particular an octadecanoid, an eicosanoid or a mixture thereof.

In the present invention, “octadecanoid” means an oxygenated molecule derived from the enzymatic or non-enzymatic oxidation of a polyunsaturated fatty acid containing 18 carbon atoms as defined above, the polyunsaturated fatty acid containing 18 carbon atoms possibly being, for example, linoleic acid, alpha-linolenic acid, gamma-linolenic acid or stearidonic acid, preferably linoleic acid, alpha-linolenic acid or gamma-linolenic acid. Examples include 9-hydroxy-10E,12Z-octadecadienoic acid (9-HODE), 13-hydroxy-9Z,11E-octadecadienoic acid (13-HODE), 9-hydroperoxy-10E,12Z-octadecadienoic acid (9-HpODE), 13-hydroperoxy-9Z,11E-octadecadienoic acid (13-HpODE), 9,10-dihydroxy-12Z-octadecadienoic acid (9,10-DiHOME), 12,13-dihydroxy-9Z-octadecadienoic acid (12,13-DiHOME), 9,12,13-trihydroxy-10E-octadecadienoic acid (10-TriHOME), 9-keto-10E,12Z-octadecadienoic acid (9-OxoODE), 13-keto-9Z,11E-octadecadienoic acid (13-OxoODE), 9-hydroxy-10E,12Z,15Z-octadecatrienoic acid (9-HOTrE), 13-hydroxy-9Z,11E,15Z-octadecatrienoic acid (13-HOTrE), 9-hydroperoxy-10E,12Z,15Z-octadecatrienoic acid (9-HpOTrE), 11-hydroperoxy-9Z,12Z,15Z-octadecatrienoic acid (11-HpOTrE), 12-hydroperoxy-9Z,13E,15E-octadecatrienoic acid (12-HpOTrE), 13-hydroperoxy-9Z,11E,15Z-octadecatrienoic acid (13-HpOTrE), 12,13-epoxy-9Z-octadecenoic acid (12,13-EpOME), or a mixture thereof.

In the present invention, “eicosanoid” means an oxygenated molecule derived from the enzymatic or non-enzymatic oxidation of a polyunsaturated fatty acid containing 20 carbon atoms as defined above, the polyunsaturated fatty acid containing 20 carbon atoms possibly being, for example, arachidonic acid or eicosapentaenoic acid. Examples include leukotrienes and/or prostanoids. Examples include 5-hydroxy-6E,8Z,11Z,14Z,17Z-eicosapentaenoic acid (5-HEPE), 12-hydroxy-5Z,8Z,10E,14Z,17Z-eicosapentaenoic acid (12-HEPE), 15-hydroxy-5Z,8Z,11Z,13E,17Z-eicosapentaenoic acid (15-HEPE), 15-hydroperoxy-5Z,8Z,11Z,13E,17Z-eicosapentaenoic acid (15-HpEPE), 5-hydroxy-6E,8Z,11Z,14Z-eicosatetraenoic acid (5-HETE), 8-hydroxy-5Z,9E,11Z,14Z-eicosatetraenoic acid (8-HETE), 9-hydroxy-5Z,7E,11Z,14Z-eicosatetraenoic acid (9-HETE), 12-hydroxy-5Z,8Z,10E,14Z-eicosatetraenoic acid (12-HETE), 15-hydroxy-5Z,8Z,11Z,13E-eicosatetraenoic acid (15-HETE), 5-hydroperoxy-6E,8Z,11Z,14Z-eicosatetraenoic acid (5-HpETE), 11-hydroperoxy-5Z,8Z,12E,14Z-eicosatetraenoic acid (11-HpETE), 12-hydroperoxy-5Z,8Z,10E,14Z-eicosatetraenoic acid (12-HpETE), 15-hydroperoxy-5Z,8Z,11Z,13E-arachidonic acid (15-HpETE), or a mixture thereof.

In the present invention, “extract enriched in oxylipins” means an extract as defined above obtained from a plant or a part thereof containing oxylipins, and whose oxylipin synthesis has been stimulated, or an extract as defined above which has undergone an oxylipin enrichment step (e.g. by chromatography).

In the present invention, “apolar solvent” means a lipophilic solvent which makes it possible to solubilize compounds of low polarity, i.e., having a Log P greater than or equal to 1, chosen, for example, from heptane, hexane, limonene, chloroform, dichloromethane, supercritical CO₂, a supercritical CO₂/ethanol mixture and mixtures of these solvents. Examples also include 100% biosourced lipophilic solvents such as, for example, EcoXtract® LIPOCOS (supplier Pennakem Europa).

In the present invention, “hydrophilic solvent” means a polar solvent chosen, for example, from water, subcritical water, water-miscible alcohols such as, for example, C1 to C3 alcohols, C3 to C5 glycols, glycerol, acetone, and mixtures thereof.

In the present invention, “organic solvent immiscible with a vegetable oil” means an organic solvent which cannot mix, or can only partially mix, with the vegetable oil, so that the mixture of the organic solvent and the vegetable oil gives a heterogeneous mixture in which at least two distinct phases can be observed.

In the present invention, “C1 to C3 alcohol” means an alcohol R2OH in which the R2 chain is a saturated, linear or branched hydrocarbon chain comprising 1 to 3 carbon atoms. Examples include methanol, ethanol, n-propanol or isopropanol, in particular methanol, ethanol or isopropanol. Preferably, it will be ethanol.

In the present invention, “C3 to C5 glycol” means a saturated, linear or branched hydrocarbon chain comprising 3 to 5 carbon atoms, the said chain carrying two OH functions. Mention may be made, for example, of propylene glycol.

In the present invention, the term “ambient temperature” means a temperature comprised between 15 and 40° C., preferably between 2° and 30° C., especially approximately 25° C.

### DETAILED DESCRIPTION OF THE INVENTION

The invention concerns an oxylipin, including a mixture of oxylipins, which may be in the form of a plant extract containing or enriched in oxylipins, for its use in the treatment of seborrhea and/or acne, preferably seborrhea. Seborrhea can be seborrhea of the skin or the scalp.

The oxylipin is as defined above and may be chosen in particular from octadecanoids, eicosanoids and mixtures thereof. Thus, the oxylipin may be chosen from 9-hydroxy-10E,12Z-octadecadienoic acid (9-HODE), 13-hydroxy-9Z,11E-octadecadienoic acid (13-HODE), 9-hydroperoxy-10E,12Z-octadecadienoic acid (9-HpODE), 13-hydroperoxy-9Z,11E-octadecadienoic acid (13-HpODE), 9,10-dihydroxy-12Z-octadecadienoic acid (9,10-DiHOME), 12,13-dihydroxy-9Z-octadecadienoic acid (12,13-DiHOME), 9,12,13-trihydroxy-10E-octadecadienoic acid (10-TriHOME), 9-keto-10E,12Z-octadecadienoic acid (9-OxoODE), 13-keto-9Z,11E-octadecadienoic acid (13-OxoODE), 9-hydroxy-10E,12Z,15Z-octadecatrienoic acid (9-HOTrE), 13-hydroxy-9Z, 11E,15Z-octadecatrienoic acid (13-HOTrE), 9-hydroperoxy-10E,12Z,15Z-octadecatrienoic acid (9-HpOTrE), 11-hydroperoxy-9Z,12Z,15Z-octadecatrienoic acid (11-HpOTrE), 12-hydroperoxy-9Z,13E,15E-octadecatrienoic acid (12-HpOTrE), 13-hydroperoxy-9Z,11E,15Z-octadecatrienoic acid (13-HpOTrE), 12,13-epoxy-9Z-octadecenoic acid (12,13-EpOME), 5-hydroxy-6E,8Z,11Z,14Z,17Z-eicosapentaenoic acid (5-HEPE), 12-hydroxy-5Z,8Z,10E,14Z,17Z-eicosapentaenoic acid (12-HEPE), 15-hydroxy-5Z,8Z,11Z,13E,17Z-eicosapentaenoic acid (15-HEPE), 15-hydroperoxy-5Z,8Z,11Z,13E,17Z-eicosapentaenoic acid (15-HpEPE), 5-hydroxy-6E,8Z,11Z,14Z-eicosatetraenoic acid (5-HETE), 8-hydroxy-5Z,9E,11Z,14Z-eicosatetraenoic acid (8-HETE), 9-hydroxy-5Z,7E,11Z,14Z-eicosatetraenoic acid (9-HETE), 12-hydroxy-5Z,8Z,10E,14Z-eicosatetraenoic acid (12-HETE), 15-hydroxy-5Z,8Z,11Z,13E-eicosatetraenoic acid (15-HETE), 5-hydroperoxy-6E,8Z,11Z,14Z-eicosatetraenoic acid (5-HpETE), 11-hydroperoxy-5Z,8Z,12E,14Z-eicosatetraenoic acid (11-HpETE), 12-hydroperoxy-5Z,8Z,10E,14Z-eicosatetraenoic acid (12-HpETE), 15-hydroperoxy-5Z,8Z,11Z,13E-arachidonic acid (15-HpETE), and a mixture thereof.

According to a particular embodiment, the oxylipin is chosen from 10-TriHOME, 13-HODE, 9-HODE, 13-OxoODE, 9-OxoODE, 12,13-DiHOME, 15-HEPE and mixtures thereof.

The oxylipin is preferably present in a plant extract, preferably an oxylipin enriched plant extract. The plant extract will contain at least one oxylipin as defined above, in particular at least one oxylipin chosen from 10-TriHOME, 13-HODE, 9-HODE, 13-OxoODE, 9-OxoODE, 12,13-diHOME, 15-HEPE and mixtures thereof. The plant extract can more particularly be an oleaginous plant extract, preferably enriched in oxylipins, which can be obtained from various oleaginous plant materials.

The oleaginous plant material is an oleaginous plant or part of a plant, i.e., rich in fat, and more particularly in vegetable oil containing polyunsaturated fatty acids. The oleaginous plant material may therefore be, for example, a vegetable oil containing polyunsaturated fatty acids. The oleaginous plant material may also be gametophytes, preferably containing polyunsaturated fatty acids.

The vegetable oil containing polyunsaturated fatty acids will more particularly contain polyunsaturated fatty acids containing 18 carbon atoms (C18) such as linoleic acid, alpha-linolenic acid or gamma-linolenic acid; polyunsaturated fatty acids containing 20 carbon atoms (C20) such as arachidonic acid or eicosapentaenoic acid; or a mixture thereof. Preferably, the content of polyunsaturated fatty acids in the vegetable oil is at least 10%, preferably at least 40% by weight relative to the total fatty acid weight of the vegetable oil. Preferably, the content of C18 and/or C20 polyunsaturated fatty acids in the vegetable oil is at least 15%, preferably at least 40% by weight relative to the total fatty acid weight of the vegetable oil.

The vegetable oil containing polyunsaturated fatty acids may be, for example, apple seed oil, soybean oil, linseed oil, safflower oil, cumin oil, rice bran oil, camelina oil, canola oil, perilla oil, chia oil, rapeseed oil, hazelnut oil or a mixture thereof. The gametophytes may be gametophytes of brown algae such as Saccharina latissima.

The oleaginous plant extract, preferably enriched in oxylipins, will more particularly be an apple seed extract (more particularly apple seed oil), a soybean extract (more particularly soybean oil), a hazelnut extract (more particularly hazelnut oil), or an extract of gametophytes of brown algae such as Saccharina latissima, said extract therefore containing oxylipins, and preferably being enriched in oxylipins.

Preferably, the oleaginous plant extract will not be a hazelnut extract such as hazelnut oil.

According to a particular embodiment of the invention, the synthesis of oxylipins can be stimulated in the plant or a part thereof (e.g. a vegetable oil or gametophytes), before extraction of the plant or a part thereof. This means that the growth or cultivation parameters of the plant or part thereof are modified in such a way as to stimulate oxylipin synthesis in the plant or part thereof and thus increase the oxylipin content in the plant or part thereof. Oxylipin synthesis can be stimulated in the plant or part thereof, for example by optimizing the parameters favoring oxidation processes, such as temperature, or by enzymatic treatment. Oxylipin synthesis can also be stimulated by elicitation, more particularly in the context of plant cell cultures, for example gametocytes.

According to another embodiment, the plant extract can be enriched in oxylipins, for example by molecular distillation of the extract and/or by fractionation of the extract, especially by chromatographic techniques. Oxylipin synthesis can be stimulated in the plant or part thereof as described above prior to extraction of the plant or part thereof.

When the plant or part thereof is a vegetable oil containing polyunsaturated fatty acids (hereinafter referred to as “vegetable oil”), the vegetable oil can be obtained by pressing the oleaginous plant raw material (e.g. grains, seeds, etc.), especially by cold pressing, i.e. without heating, at ambient temperature, preferably followed by a filtration step.

Vegetable oil can also be obtained by extracting the oleaginous plant raw material (e.g. grains, seeds, etc.), whole or ground, with an apolar solvent. Such extraction can be carried out with supercritical CO₂, with or without the addition of ethanol as co-solvent, and optionally followed by a filtration step.

The vegetable oil can also be obtained by extraction of the oleaginous vegetable raw material with a hydrophilic solvent in the presence of at least one enzyme (e.g. pectinase), and especially of an enzymatic mixture such as pectinases for example.

Preferably, the vegetable oil will be obtained by pressing the oleaginous plant raw material, especially by cold pressing, i.e. without heating, at ambient temperature, preferably followed by a filtration step.

The oleaginous plant extract can be obtained from vegetable oil, by extraction with an aqueous hydrotropic solution, subcritical water or an organic solvent immiscible with vegetable oil, optionally mixed with water. The oleaginous plant extract can be enriched in oxylipins, for example by molecular distillation of the extract, or by fractionating the extract by techniques. The organic solvent immiscible with vegetable oil may be used as a mixture with water, especially in a volume ratio of organic solvent/water comprised between 80/20 and 100/0, especially comprised between 85/15 and 95/5, in particular approximately 90/10.

The organic solvent immiscible with vegetable oil may especially be a C1 to C3 alcohol, optionally mixed with water, in particular in the organic solvent/water volume ratio described above. The C1 to C3 alcohol will more particularly be methanol, ethanol, n-propanol or isopropanol, in particular methanol, ethanol or isopropanol, preferably ethanol.

The extraction solvent may especially be chosen from methanol, a methanol/water mixture, ethanol, an ethanol/water mixture, isopropanol and an isopropanol/water mixture. According to a preferred embodiment, the extraction solvent will be methanol, an ethanol/water mixture in a volume ratio of approximately 90/10 or an isopropanol/water mixture in a volume ratio of approximately 90/10.

According to a preferred embodiment, the oleaginous plant extract can be obtained by a method according to the invention described below.

A method for preparing an oleaginous plant extract comprises a step of extracting a vegetable oil with an extraction solvent comprising, especially composed of, a hydrotropic aqueous solution, subcritical water, or an organic solvent immiscible with vegetable oil, optionally mixed with water.

According to a particular embodiment, the extraction solvent comprises, especially is composed of, an organic solvent immiscible with vegetable oil, optionally mixed with water. The organic solvent immiscible with vegetable oil may especially be a C1-C3 alcohol.

The extraction solvent may especially be a C1 to C3 alcohol optionally mixed with water.

The C1 to C3 alcohol may be methanol, ethanol, n-propanol or isopropanol, in particular methanol, ethanol or isopropanol. Preferably, it will be ethanol.

The organic solvent immiscible with vegetable oil, in particular a C1 to C3 alcohol such as methanol, ethanol or isopropanol, may be used as a mixture with water, especially in an organic solvent/water volume ratio comprised between 80/20 and 100/0, especially comprised between 85/15 and 95/5, in particular approximately 90/10.

The extraction solvent may especially be chosen from methanol, a methanol/water mixture, ethanol, an ethanol/water mixture, isopropanol and an isopropanol/water mixture.

According to a preferred embodiment, the extraction solvent will be methanol, an ethanol/water mixture in a volume ratio of approximately 90/10 or an isopropanol/water mixture in a volume ratio of approximately 90/10.

The vegetable oil extraction step will be carried out in particular by mixing the vegetable oil with the extraction solvent for 1 to 12 h and in particular at a temperature comprised between 15 and 25° C., especially approximately 20° C. The quantity of extraction solvent used to carry out this extraction will advantageously be from 0.5 to 3 g, in particular from 1 to 3 g per 1 g of vegetable oil.

An extraction phase and a lipid phase will then be obtained at the end of this extraction. The extraction phase will advantageously be separated from the lipid phase and recovered before being dried, partially or totally, especially under vacuum, to more or less remove the extraction solvent and obtain either the dry extract if the solvent is completely removed, or the concentrated extract which is diluted in residual solvent.

According to one embodiment according to the invention, the method according to the invention will comprise the following two successive steps:
1. obtaining a vegetable oil, and
2. extracting the vegetable oil with an extraction solvent, comprising, especially composed of, an aqueous hydrotropic solution, subcritical water or an organic solvent immiscible with vegetable oil, optionally mixed with water.

According to a preferred embodiment according to the invention, the method according to the invention will comprise the following successive steps:
1. optionally obtaining a vegetable oil,
2. extracting the vegetable oil with an extraction solvent, comprising, especially composed of, an aqueous hydrotropic solution, subcritical water or an organic solvent immiscible with oil from plant achenes optionally mixed with water, to give an extraction phase and a lipid phase,
3. recovery of the extraction phase obtained in step (2), and
4. partial or total drying of the extraction phase to give a concentrated or dry extract according to the invention.

Step (1) can be carried out by pressing the oleaginous plant raw material (e.g. grains, seeds, etc.), especially by cold pressing, i.e., without heating, at ambient temperature, preferably followed by a filtration step.

Step (2) will advantageously be carried out with an extraction solvent as defined above.

The extraction solvent may be methanol, ethanol or isopropanol, optionally mixed with water, especially in an organic solvent/water volume ratio comprised between 80/20 and 100/0, especially comprised between 85/15 and 95/5, in particular approximately 90/10. An advantageous extraction solvent is an ethanol/water mixture in a volume ratio of approximately 90/10.

The extraction step (2) can be carried out by mixing vegetable oil with the extraction solvent for 1 to 12 h and, in particular, at a temperature of 15 to 25° C., especially approximately 20° C. The quantity of extraction solvent used to carry out this extraction will advantageously be from 0.5 to 3 g, in particular from 1 to 3 g per 1 g of vegetable oil. This extraction step (2) makes it possible to obtain at the end an extraction phase of interest and a lipid phase.

Step (3) will advantageously be carried out by separating the extraction phase from the lipid phase.

Step (4) will advantageously be carried out under vacuum.

The extract enriched in oxylipins will contain in particular at least 25% by weight, for example at least 30% by weight, for example at least 40% by weight, for example at least 50% by weight, for example at least 60% by weight, for example at least 70% by weight, for example at least 80% by weight, for example at least 90% by weight, of oxylipins relative to the weight of dry extract. The extract enriched in oxylipins may comprise up to 100% by weight of oxylipins.

The present invention also concerns a dermo-cosmetic or dermatological composition comprising at least one oxylipin as described above, with at least one dermo-cosmetically or dermatologically acceptable excipient, for the treatment of seborrhea and/or acne, preferably seborrhea. Seborrhea can be seborrhea of the skin or the scalp.

The oxylipin will more particularly be derived from a plant and more particularly from an oleaginous plant material and may be present in a plant extract as defined above, and more particularly a plant extract enriched in oxylipins. The plant extract may more particularly be an oleaginous plant extract as defined above, preferably enriched in oxylipins. The oleaginous plant extract will more particularly be an apple seed extract (more particularly apple seed oil), a soybean extract (more particularly soybean oil), a hazelnut extract (more particularly hazelnut oil), or an extract of gametophytes of brown algae such as Saccharina latissima, said extract therefore containing oxylipins.

The dermo-cosmetic or dermatological composition according to the invention is typically in a form suitable for topical use. The dermo-cosmetic or dermatological composition according to the invention is therefore not intended to be rinsed after application (typically, the skin is not rinsed or washed at least 12 hours after application of the composition). The dermo-cosmetic or dermatological compositions may thus be provided in the forms which are usually known for topical administration, i.e., especially lotions, milks, emulsions, serums, balms, masks, creams, dispersions, gels, foams, sprays and shampoos.

The dermo-cosmetic or dermatological compositions may also contain surfactants, complexing agents, preservatives, antioxidants such as tocopherols, stabilizers, emulsifiers, thickeners, gelling agents, humectants, emollients, trace elements, essential oils, fragrances, dyes, matting agents, chemical or mineral sunscreen agents, moisturizing agents, thermal waters, etc.

The examples that follow, as well as the figures, illustrate the invention without limiting its scope.

### FIGURES

FIG. 1: High performance liquid chromatography (HPLC) chromatogram of a lipid extract of Saccharina latissima gametophytes (UV 236 nm) showing the presence of molecules with the UV signature characteristic of oxylipins, and in particular of 12(S)-HEPE and 15(S)-HEPE. The oxylipin 15(S)HEPE is the most abundant.

FIG. 2: Production of oxylipins of gametophytes of Saccharina latissima suspended in a culture medium for 48 h without (control) or with addition of phosphates (75 mg/L K₂HPO₄+50 mg/L β-glycerophosphate Na₂×H₂O) and/or iron Fe³⁺ (0.91 mg/L FeCl₃·6H₂O) into the culture medium. The oxylipin contents are expressed in mg equivalent 15(S)HEPE per liter of suspension.

FIG. 3: Evolution over time of the quantities of oxylipins produced (in mg equivalent 15(S)HEPE) per liter of culture medium for cultures A (control) and B (culture medium enriched in phosphates).

FIG. 4: Evolution over time of the quantities of oxylipins (in mg equivalent 15(S)HEPE) produced per liter of culture medium for culture A (control) and culture B (elicited by phosphate and iron). Elicitation (indicated by an arrow) is carried out on the 34th day of culture by the addition of phosphates (75 mg/L K₂HPO₄+50 mg/L β-glycerophosphate Na₂×H₂O) and iron Fe³⁺ (1 mg/L FeCl₃·6H₂O) in the culture medium.

### EXAMPLES

### Example 1: Lipid Extract Obtained from Apple Seed Oil

The lipid extract was obtained as follows:
Cold pressing of apple seeds (Pyrus malus L.) to obtain an apple seed oil whose fatty acid fraction shows a linoleic acid content of 50% by weight;
Extraction of apple seed oil by ethanol/water mixture (90/10 v/v) with 2 grams of ethanol/water mixture per gram of oil for 2 hours at 20° C.;
Recovery of the ethanol phase, and evaporation of the solvent under vacuum to obtain a lipid extract.

The lipid extract was obtained with an extraction mass yield of approximately 1% for which the main octadecanoids are 10-TriHOME, 13-HODE, 9-HODE, 13-OxoODE and 9-OxoODE.

### Example 2: Lipid Extract Obtained from Soybean Oil

The lipid extract was obtained as follows:
Cold pressing of soybeans (Glycine max (L.) Merr.) to obtain a soybean oil whose fatty acid fraction shows a content of 55% linoleic acid and 8% alpha-linolenic acid;
Extraction of soybean oil by ethanol/water mixture (90/10 v/v) with 2 grams of ethanol/water mixture per gram of oil for 2 hours at 20° C.;
Recovery of the ethanol phase, and evaporation of the solvent under vacuum to obtain a lipid extract.

The lipid extract was obtained with an extraction mass yield of approximately 5.5% for which the main octadecanoids are 10-TriHOME, 13-HODE, 9-HODE, 13-OxoODE and 9-OxoODE.

### Example 3: Lipid Extract Obtained from Hazelnut Oil

The lipid extract was obtained as follows:
Cold pressing of shelled hazelnuts (Corylus avellana L.) to obtain a hazelnut oil whose fatty acid fraction shows a linoleic acid content of 15%;
Extraction of hazelnut oil by ethanol/water mixture (90/10 v/v) with 2 grams of ethanol/water mixture per gram of oil for 2 hours at 20° C.;
Recovery of the ethanol phase, and evaporation of the solvent under vacuum to obtain a lipid extract.

The lipid extract was obtained with an extraction mass yield of approximately 1.5% and whose main octadecanoids are 10-TriHOME, 13-HODE, 9-HODE, and 12,13-DiHOME.

### Example 4: Lipid Extract Obtained from Saccharina latissima

4a: Experiment in a Culture Medium with or without Addition of Phosphates and/or Ferric Iron

A strain of male gametophyte of Saccharina latissima is cultivated in reconstituted seawater. The biomass cultures are carried out in a bioreactor at 18° C. with a supply of air enriched in CO₂ so as to maintain a pH at 8.0 and pneumatic stirring of the suspension. The culture is illuminated by light-emitting diodes over a photoperiod of 16/8 h. Once the biomass has reached a critical density, the gametophyte biomass is collected on a nylon sieve and lyophilized under vacuum just before solid/liquid extraction. This is done by grinding in the presence of solvent (e.g. a 52:48 ethanol/methyl ethyl ketone mixture (v/v)). Grinding is carried out using a vibrator (Retsch MM 400) or alternatively an ultrasonic mill (Branson, Digital Sonifier 450). Between each grinding, the tubes containing the biomasses are centrifuged at 8000 g for 10 min. The clarified supernatants are collected and then fresh solvent is introduced into the grinding tubes containing the biomass pellets for a second repetition of the grinding steps. The collected supernatant fractions undergo a decolorization step. For this, the fractions are diluted in purified water and incubated at 4° C. in the dark. The tubes are then centrifuged and the decolorized supernatants are collected. Then a solvent support (DMSO, pentylene glycol, Eutanol® G, etc.) is placed in the decolorized fractions so as to ensure a final dry matter content after complete evaporation of the solvents and water, leaving only the dry matter solubilized in the chosen support.

The analysis is carried out by HPLC (Alliance WATERS 2695 system controlled by Empower Ver 2.03 software equipped with a WATERS 2996 diode array detector) using a CORTECS C8 2.7μ 4.6×150 mm column equipped with a CORTECS C8 2.7μ 3.9×5 mm VG guard column inserted into a furnace at 35° C. The mobile phases are A=distilled water with 0.1% formic acid; B=acetonitrile with 0.1% formic acid; C=methanol with 0.1% formic acid. The flow rate is 0.7 mL/min with the following gradient: 1.5 min 50% A, 50% B; 22 min 30% A, 70% B; 25 min 30% A, 70% B; 25.1 min 100% C; 29 min 100% C; 29.1 min 50% A, 50% B; 35 min 50% A, 50% B. UV detection is carried out at 236.5 nm. The device is calibrated by successive injections of a solution of 15(S)-HEPE (Cayman Chemical) at 10 mg/L and giving a linear correlation between the areas detected and the quantities analyzed.

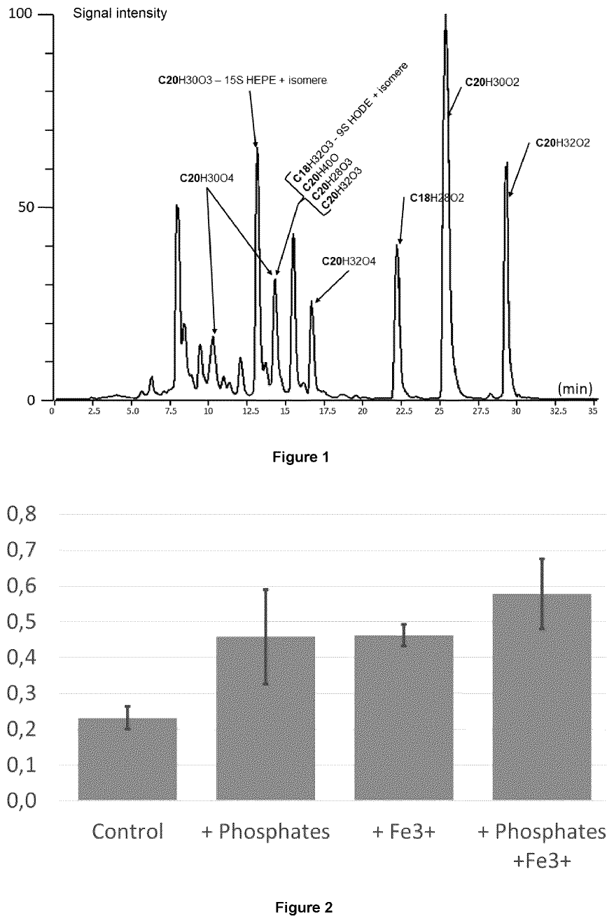

The chromatograms show the presence of several isomers and derived oxylipins having the UV signature characteristic of oxylipins identical to that of 15(S)-HEPE (FIG. 1). These form peaks at retention times ranging from 10 minutes to 30 minutes and oxylipin 15(S)-HEPE, which has the most abundant peak, has a retention time of 13 minutes. The oxylipin content is expressed in equivalents μg 15(S)-HEPE by summing the areas of the 15(S)-HEPE peaks and isomers and the derived oxylipins.

Analysis of samples from control and elicited biomasses after addition of phosphates (75 mg/L K₂HPO₄+50 mg/L β-glycerophosphate Na₂×H₂O) and/or iron Fe³⁺ (0.91 mg/L FeCl₃·6H2O) into the culture medium made it possible to observe that the overall production of oxylipins per liter of suspension from the control culture is 0.23 mg/L while cultures elicited using phosphates or ferric iron (Fe³⁺) have similar overall production of oxylipins of around 0.46 mg/L. The simultaneous addition of phosphates and ferric iron makes it possible to observe overall oxylipin production per liter of suspension of 0.58 mg/L (FIG. 2).

### 4b: Experiment of Elicitation of the Culture in a Bioreactor by Addition of Phosphates at the Beginning of the Culture

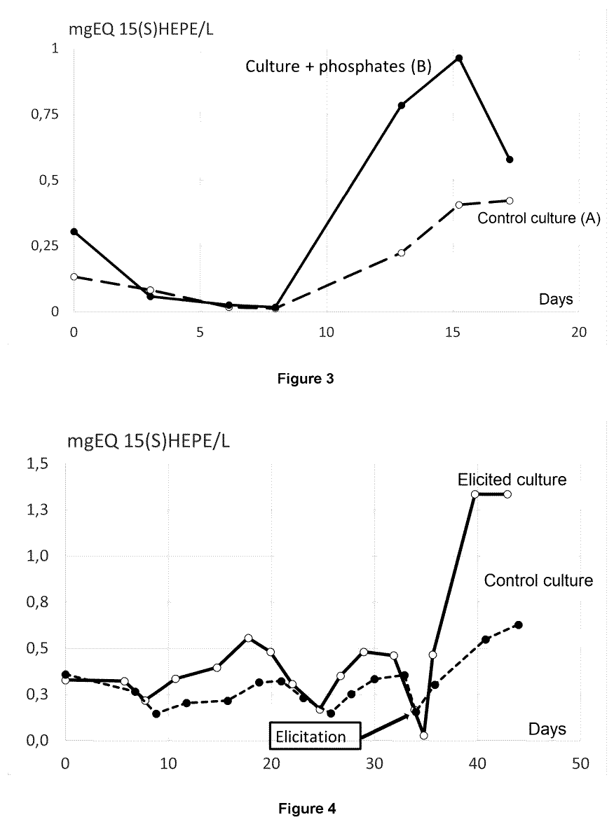

A culture of male gametophyte of Saccharina latissima is carried out in a 10-L bioreactor for 63 days. Five L of this culture is transferred into another bioreactor in order to produce two parallel cultures A and B. The two cultures A and B receive 5 L of fresh medium previously sterilized and cooled. A further addition of phosphates is carried out only into culture B. Thus, the phosphate concentrations for culture A are 37.5 mg/L of K₂HPO₄ and 25 mg/L of β-glycerophosphate Na₂×H₂O supplied by the culture medium and for culture B 112.5 mg/L of K₂HPO₄ and 75 mg/L of β-glycerophosphate Na₂×H₂O supplied by the culture medium and the further addition. The two cultures are then carried out identically with the same lighting, temperature and pH control conditions. The biomasses are harvested and then frozen at −20° C. at regular intervals. HPLC analysis of total oxylipins expressed in mg of 15(S)-HEPE equivalent per liter of culture shows that from the 10^{th} day of culture, the contents of the culture that received the most phosphates (B) are greater than those of the control culture (A); this difference is at its peak on the 15^{th} day of culture (FIG. 3).

### 4c: Experiment of Elicitations of the Culture in a Bioreactor by Addition of Phosphates and Iron During Culture

A culture of male gametophyte of Saccharina latissima is carried out in a 10-L bioreactor for 63 days. Five L of this culture is transferred into another bioreactor in order to produce two parallel cultures A and B. The two cultures A and B receive 5 L of fresh medium previously sterilized and cooled. The two cultures are then carried out identically for 34 days with the same lighting, temperature and pH control conditions. Phosphates and ferric iron are added on day 34 only to culture B (elicited culture) by adding 75 mg/L of K₂HPO₄, 50 mg/L of β-glycerophosphate Na₂×H₂O and 1 mg/L of FeCl₃·6H₂O. The biomasses are harvested and then frozen at −20° C. at regular intervals. HPLC analysis of total oxylipins expressed in mg of 15(S)-HEPE equivalent per liter of culture shows that from day 36, i.e. 48 hours after elicitation, the contents of the culture that received elicitation (B) are greater than those of the control culture (A); this difference is observable until the end of the experiment (FIG. 4).

### Example 5: Effects of Different Plant Extracts Containing Oxylipins on Lipogenesis Induced by Arachidonic Acid in Sebocytes

The sebocyte is the predominant cell type of the sebaceous glands. They are differentiated epithelial cells that gradually accumulate lipids and release their contents through cellular degradation and cell membrane disruption, this process being known as holocrine secretion. During differentiation, sebocytes accumulate large amounts of lipids in cytoplasmic vesicles in the form of lipid droplets. Sebocyte differentiation is modulated by several signaling pathways, such as the PPAR-g dependent pathway or the COX pathway.

### Method

In this study, a model of sebocytes derived from induced pluripotent stem cells from 2 different donors is used. These cells are treated with arachidonic acid to specifically induce lipogenesis. The modulating activity of different plant extracts is therefore evaluated on lipogenesis in sebocytes.

The lipid extract derived from apple seed oil is prepared according to Example 1.

The lipid extract derived from soybean oil is prepared according to Example 2.

The lipid extract derived from hazelnut oil is prepared according to Example 3.

The lipid extract derived from the gametophytes of Saccharina latissima is prepared according to Example 4a.

All these extracts are solubilized in DMSO (dimethyl sulfoxide) and are tested at 2 different concentrations.

The sebocytes derived from induced pluripotent stem cells originating from 2 different donors are incubated for 3 days with a specific molecule (PhenoCULT® NPC PHENOCELL, Grasse, France) in order to accelerate their maturation before the tests. Lipogenesis of the sebocytes is induced by arachidonic acid (10 μM) for 48 hours in the presence or absence of the lipid extracts to be tested.

The results are generated from 3 independent experiments, but done with replicates, to obtain n>4.

Quantification of lipid synthesis is evaluated after Bodipy staining with high resolution imaging and normalized with Hoechst staining (nuclei labeling).

The inter-group comparison is carried out by one-way ANOVA followed by Tukey's test.

### Results

The addition of arachidonic acid (AA) results in a significant accumulation of total lipids in human sebocytes after 48 hours. These results are summarized in Table 1 below.

TABLE 1

| | | | AA (10 μM) + |
|---|---|---|---|
| | | | Reference product |
| Total lipids | Control | AA (10 μM) | (1 μM) |
| Mean ± sem | 80.3 ± 4.7 | 1537.6 ± 593.7 | 144.0 ± 23.1 |

AA: arachidonic acid;
sem: standard error of the mean

The cannabidiol compound, a reference product in the inhibition of lipogenesis, acts by activating the TRPV4 ion channels (Olah et al., 2014). It is tested at 1 μM (also dissolved in DMSO); it significantly inhibits lipid accumulation. This result was expected; it validates the test (Table 1).

### Lipid Extract of Apple Seed Oil

The results obtained with the lipid extract of apple seed oil are presented in Table 2.

TABLE 2

| | | AA (10 μM) + | AA (10 μM) + |
|---|---|---|---|
| | | Apple seed oil | Apple seed oil |
| | AA | extract | extract |
| Total lipids | (10 μM) | (5 μg/mL) | (50 μg/mL) |
| Mean ± sem | 1684.5 ± 485.2 | 258.9 ± 87.0 | 290.7 ± 122.4 |
| Fold change | — | −6.7 | −6.5 |
| vs AA | | | |

AA: arachidonic acid;
sem: standard error of the mean

The lipid extract of apple seed oil, enriched in oxylipins, at the two concentrations tested, significantly decreased (p<0.001 versus the arachidonic acid group) the total lipids (Table 2). Indeed, the fold change makes it possible to appreciate the variations between different groups, a negative value indicating an inhibition and a value less than −2 indicating a reduction of more than 50%.

The inventors clearly demonstrate that a lipid extract of apple seed oil, enriched in oxylipins, especially the main octadecanoids (10-TriHOME, 13-HODE, 9-HODE, 13-OxoODE, 9-OxoODE) induces a significant and very important inhibition of lipogenesis induced by arachidonic acid in human sebocytes, at the two concentrations tested.

### Lipid Extract of Soybean Oil

The results obtained with the lipid extract of soybean oil are presented in Table 3.

TABLE 3

| | | AA (10 μM) + | AA (10 μM) + |
|---|---|---|---|
| | | Soybean | Soybean |
| | AA | oil extract | oil extract |
| Total lipids | (10 μM) | (50 μg/mL) | (100 μg/mL) |
| Mean ± sem | 6813.0 ± 5666.2 | 1931.9 ± 1507.3 | 1267.9 ± 798.6 |
| Fold change | — | −3.0 | −5.4 |
| vs AA | | | |

AA: arachidonic acid;
sem: standard error of the mean

The lipid extract of soybean oil, enriched in oxylipins, at the two concentrations tested, significantly decreased (p<0.01 for the two concentrations tested) the total lipids (Table 3). Indeed, the fold change makes it possible to demonstrate a reduction of much greater than 50% in total lipids.

The inventors clearly demonstrate that a lipid extract of soybean oil, enriched in oxylipins, especially the main octadecanoids (10-TriHOME, 13-HODE, 9-HODE, 13-OxoODE, 9-OxoODE) induces a significant and very important inhibition of lipogenesis induced by arachidonic acid in human sebocytes, at the two concentrations tested.

### Lipid Extract of Hazelnut Oil

The results obtained with the lipid extract of hazelnut oil are presented in Table 4.

TABLE 4

| | | AA (10 μM) + | AA (10 μM) + |
|---|---|---|---|
| | | Hazelnut | Hazelnut |
| | AA | oil extract | oil extract |
| Total lipids | (10 μM) | (5 μg/mL) | (50 μg/mL) |
| Mean ± sem | 6813.0 ± 5666.2 | 2385.1 ± 2119.5 | 1058.2 ± 435.2 |
| Fold change | — | −3.8 | −5.1 |
| vs AA | | | |

AA: arachidonic acid;
sem: standard error of the mean

The lipid extract of hazelnut oil, enriched in oxylipins, at the two concentrations tested, significantly decreased (p<0.001 versus the arachidonic acid group) the total lipids (Table 4). Indeed, the fold change reaches −3.8 at the lowest concentration tested, revealing a more than 3-fold reduction in total lipids.

The inventors clearly demonstrate that an extract of hazelnut oil, enriched in oxylipins, especially the main octadecanoids (10-TriHOME, 13-HODE, 9-HODE, 12,13-DiHOME) induces a significant and very important inhibition of lipogenesis induced by arachidonic acid in human sebocytes, at the two concentrations tested.

Extract of Saccharina latissima

The results obtained with the Saccharina latissima extract are presented in Table 5.

TABLE 5

| | | AA (10 μM) + | AA (10 μM) + |
|---|---|---|---|
| | | S. laminaria | S. laminaria |
| | AA | extract | extract |
| Total lipids | (10 μM) | (3 μg/mL) | (10 μg/mL) |
| Mean ± sem | 6754.2 ± 4890.2 | 1970.2 ± 1553.9 | 404.0 ± 143.9 |
| Fold change | — | −3.6 | −12.5 |
| vs AA | | | |

AA: arachidonic acid;
sem: standard error of the mean

The extract of Saccharina latissima, enriched in oxylipins, at the two concentrations tested, significantly decreased (p<0.001 versus the arachidonic acid group) the total lipids (Table 5). Indeed, the fold change makes it possible to demonstrate a more than 3-fold reduction in total lipids at 3 μg/mL Saccharina latissima extract and a more than 12-fold reduction in total lipids at a concentration of 10 μg/mL Saccharina latissima extract.

The inventors clearly demonstrate that an extract of Saccharina latissima, enriched in oxylipins, especially eicosanoids such as 15(S)-HEPE, induces a significant and substantial inhibition of lipogenesis induced by arachidonic acid in human sebocytes, at the two concentrations tested.

Consequently, all these results show that all these plant extracts enriched in oxylipins have the ability to inhibit lipid accumulation in sebocytes, significantly reducing sebum production. It is interesting to note that very different plant extracts are capable of exhibiting anti-seborrheic activity.

### Example 6: Fractionation of a Soybean Oil Extract Obtained According to Example 2

### Protocol 1: Fractionation Conditions

The separation is carried out on a Chromabond™ Flash RS 80 SiOH 40-63 μm column (Macherey-Nagel™ brand), column volume 145 mL, packing volume 210 mL.
Mobile phase supply is done using a pump with a flow rate of 5 mL/min.
14.6 g of soybean oil extract obtained according to Example 2 are solubilized in 40 mL of heptane.
The solution obtained is deposited at the top of the column by the pump at 5 mL/min.
The column is then eluted with 400 mL of heptane.
The column is then eluted with 800 mL of a heptane-dichloromethane mixture (1:1) (v/v).
The column is then eluted with 500 mL of dichloromethane.
The column is then eluted with 500 mL of a dichloromethane-methanol mixture (1:1) (v/v).
The column is then eluted with 200 mL of methanol.

### Protocol 2: UPLC-DAD Analytical Conditions

Column: BEH Shield 1.7 μm C18.150*2.1 mm
Mobile phase:
   A: water+0.1% formic acid
   B: acetonitrile+0.1% formic acid
Gradient: according to Table 6 below

TABLE 6

| T (min) | A (%) | B (%) |
|---|---|---|
| 0 | 50 | 50 |
| 1 | 50 | 50 |
| 10 | 0 | 100 |
| 15 | 0 | 100 |
| 16 | 50 | 50 |
| 20 | 50 | 50 |

Column temperature: 50° C.
Flow rate: 0.4 mL/min
Detection: 205 nm and 234 nm
Injection volume: 1 μL

### Protocol 3: CPG-MS Analytical Conditions

Column: DB-5HT (Agilent Technologies); 30 m×0.32 mm×0.1 μm
Injection: 320° C.; Mode=Split
Furnace: Temperature gradient (° C.) according to Table 7 below

TABLE 7

| T (min) | Temp (°C) |
|---|---|
| 0 | 160 |
| 30 | 370 |
| 40 | 370 |

Vector gas flow rate: 1 mL/min
Detection: MS Transferline Temperature=350° C.; Ion Source Temperature=320° C.; MS-EI; Full Scan Start Mass=40; Full Scan End Mass=800
Injection volume: 1 μL

### Results

The fractions obtained are collected at the column outlet and analyzed by ultra-high pressure liquid chromatography coupled to a diode array detector (UPLC-DAD) as well as by gas chromatography coupled to a mass spectrometer (GC-MS). They are then collected according to their composition and dried by evaporation under vacuum.

This fractionation makes it possible to obtain 4 fractions with, in order of increasing polarity, triglycerides, diglycerides, and monoglycerides (Fraction 1-47% by mass), free fatty acids (Fraction 2-11% by mass), phytosterols (Fraction 3-9% by mass), then oxylipins (Fraction 4-8% by mass).

These different fractions were tested according to the method of Example 5.

Table 8 below summarizes the results obtained with a soybean oil lipid extract prepared according to Example 2 and the different fractions obtained according to Example 6 and tested at 5 μg/mL on the production of total lipids induced by arachidonic acid (AA) at 10 μM.

TABLE 8

| | AA (10 μM) + Extract/fraction (10 μM) |
|---|---|
| Extract/Fractions | Fold change vs AA |
| Soybean oil extract (Example 2), | −29.2 |
| 50 μg/mL | |
| Fraction 2 (free fatty acids), | −1.4 |
| 5 μg/mL | |
| Fraction 3 (sterols), | −2.2 |
| 5 μg/mL | |
| Fraction 4 (oxylipins)*, | −97.5 |
| 5 μg/mL | |

*Corresponds to an extract enriched in oxylipins according to the invention

The strong seboregulatory effect of soybean oil is found in this test.

Free fatty acid and sterol fractions showed little or no inhibition of lipid induction by arachidonic acid stimulated sebocytes.

Fraction 4 with oxylipins resulted in a much greater decrease than the unfractionated soybean oil extract in arachidonic acid-induced total lipid accumulation, clearly indicating that the oxylipin fraction (oxidized fatty acids) of soybean oil extract is capable of strongly inhibiting arachidonic acid-induced lipogenesis.

### REFERENCES

FR2789085
Andreou et al., 2009, Lipids, 44: 207-215
Blée, 2002, Trends in Plant Science Vol 7, No. 7, 315-321
Calder, 2012, J. Nutr. 142: 592S-599S
Cuvelier et al., 2004, Ann. Med. Vet. 148, 133-140
Lagarde, 2011, OCL, vol 18, No. 2 March-April, doi.org/10.1051/ocl.2011.0377
Liu et al., 2015, Brain Res. 0: 220-246
Oenel et al., 2017, Plant Cell Physiol. 58(5): 925-933
Olah et al., 2014, J. Clin. Invest. 124(9), 3713-3724
Richardson et al., 2017, J. Agric. Food Chem., 65: 1941-1951
Rustérucci et al., 1999, J Biol. Chem. Vol 274, No 51: 36446-36455
Van Ginneken et al., 2011, Lipids in Health and Disease, 10, 104

1.-15. (canceled)

16. A method for treating seborrhea comprising administering to a person in need thereof an effective quantity of at least one oxylipin.

17. The method according to claim 16, wherein is the at least one oxylipin is a mixture of oxylipins.

18. The method according to claim 17, wherein the mixture of oxylipins is a plant extract containing or enriched in oxylipins.

19. The method according to claim 16, wherein the seborrhea is that of the skin or scalp.

20. The method according to claim 16, wherein the at least one oxylipin is chosen from octadecanoids, eicosanoids and mixtures thereof.

21. The method according to claim 16, wherein the at least one oxylipin is at least one of 9-hydroxy-10E,12Z-octadecadienoic acid (9-HODE), 13-hydroxy-9Z,11E-octadecadienoic acid (13-HODE), 9-hydroperoxy-10E,12Z-octadecadienoic acid (9-HpODE), 13-hydroperoxy-9Z,11E-octadecadienoic acid (13-HpODE), 9,10-dihydroxy-12Z-octadecenoic acid (9,10-DiHOME), 12,13-dihydroxy-9Z-octadecenoic acid (12,13-DiHOME), 9,12,13-trihydroxy-10E-octadecenoic acid (10-TriHOME), 9-keto-10E,12Z-octadecadienoic acid (9-OxoODE), 13-keto-9Z,11E-octadecadienoic acid (13-OxoODE), 9-hydroxy-10E,12Z,15Z-octadecatrienoic acid (9-HOTrE), 13-hydroxy-9Z,11E,15Z-octadecatrienoic acid (13-HOTrE), 9-hydroperoxy-10E,12Z,15Z-octadecatrienoic acid (9-HpOTrE), 11-hydroperoxy-9Z,12Z,15Z-octadecatrienoic acid (11-HpOTrE), 12-hydroperoxy-9Z,13E,15E-octadecatrienoic acid (12-HpOTrE), 13-hydroperoxy-9Z,11E,15Z-octadecatrienoic acid (13-HpOTrE), 12,13-epoxy-9Z-octadecenoic acid (12,13-EpOME), 5-hydroxy-6E,8Z,11Z,14Z,17Z-eicosapentaenoic acid (5-HEPE), 12-hydroxy-5Z,8Z,10E,14Z,17Z-eicosapentaenoic acid (12-HEPE), 15-hydroxy-5Z,8Z,11Z,13E,17Z-eicosapentaenoic acid (15-HEPE), 15-hydroperoxy-5Z,8Z,11Z,13E,17Z-eicosapentaenoic acid (15-HpEPE), 5-hydroxy-6E,8Z,11Z,14Z-eicosatetraenoic acid (5-HETE), 8-hydroxy-5Z,9E,11Z,14Z-eicosatetraenoic acid (8-HETE), 9-hydroxy-5Z,7E,11Z,14Z-eicosatetraenoic acid (9-HETE), 12-hydroxy-5Z,8Z,10E,14Z-eicosatetraenoic acid (12-HETE), 15-hydroxy-5Z,8Z,11Z,13E-eicosatetraenoic acid (15-HETE), 5-hydroperoxy-6E,8Z,11Z,14Z-eicosatetraenoic acid (5-HpETE), 11-hydroperoxy-5Z,8Z,12E,14Z-eicosatetraenoic acid (11-HpETE), 12-hydroperoxy-5Z,8Z,10E,14Z-eicosatetraenoic acid (12-HpETE), 15-hydroperoxy-5Z,8Z,11Z,13E-arachidonic acid (15-HpETE), and mixtures thereof.

22. The method according to claim 21, wherein the at least one oxylipin is chosen from 10-TriHOME, 13-HODE, 9-HODE, 13-OxoODE, 9-OxoODE, 12,13-DiHOME, 15-HEPE and mixtures thereof.

23. The method according to claim 16, wherein the oxylipin is present in an oleaginous plant extract enriched in oxylipins.

24. The method according to claim 23, wherein the oleaginous plant extract enriched in oxylipins is an extract of apple seeds, soybeans, hazelnuts, or gametophytes of brown algae.

25. The method according to claim 24, wherein the brown algae is Saccharina latissima enriched in oxylipins.

26. The method according to claim 24, wherein the oleaginous plant extract enriched in oxylipins contains at least 25% by weight of oxylipins, relative to the weight of dry extract.

27. The method according to claim 26, wherein the oleaginous plant extract enriched in oxylipins contains at least 30% by weight of oxylipins, relative to the weight of dry extract.

28. The method according to claim 27, wherein the oleaginous plant extract enriched in oxylipins contains at least 40% by weight of oxylipins, relative to the weight of dry extract.

29. The method according to claim 28, wherein the oleaginous plant extract enriched in oxylipins contains at least 50% by weight of oxylipins, relative to the weight of dry extract.

30. A method for treating seborrhea comprising administering to a person in need thereof a dermo-cosmetic or dermatological composition comprising at least one oxylipin with at least one dermo-cosmetically or dermatologically acceptable excipient.

31. The method according to claim 30, wherein the seborrhea is that of the skin or scalp.

32. The method according to claim 30, wherein the at least one oxylipin is chosen from octadecanoids, eicosanoids and mixtures thereof.

33. The method according to claim 30, wherein the at least one oxylipin is at least one of 9-hydroxy-10E,12Z-octadecadienoic acid (9-HODE), 13-hydroxy-9Z,11E-octadecadienoic acid (13-HODE), 9-hydroperoxy-10E,12Z-octadecadienoic acid (9-HpODE), 13-hydroperoxy-9Z,11E-octadecadienoic acid (13-HpODE), 9,10-dihydroxy-12Z-octadecenoic acid (9,10-DiHOME), 12,13-dihydroxy-9Z-octadecenoic acid (12,13-DiHOME), 9,12,13-trihydroxy-10E-octadecenoic acid (10-TriHOME), 9-keto-10E,12Z-octadecadienoic acid (9-OxoODE), 13-keto-9Z,11E-octadecadienoic acid (13-OxoODE), 9-hydroxy-10E,12Z,15Z-octadecatrienoic acid (9-HOTrE), 13-hydroxy-9Z,11E,15Z-octadecatrienoic acid (13-HOTrE), 9-hydroperoxy-10E,12Z,15Z-octadecatrienoic acid (9-HpOTrE), 11-hydroperoxy-9Z,12Z,15Z-octadecatrienoic acid (11-HpOTrE), 12-hydroperoxy-9Z,13E,15E-octadecatrienoic acid (12-HpOTrE), 13-hydroperoxy-9Z,11E,15Z-octadecatrienoic acid (13-HpOTrE), 12,13-epoxy-9Z-octadecenoic acid (12,13-EpOME), 5-hydroxy-6E,8Z,11Z,14Z,17Z-eicosapentaenoic acid (5-HEPE), 12-hydroxy-5Z,8Z,10E,14Z,17Z-eicosapentaenoic acid (12-HEPE), 15-hydroxy-5Z,8Z,11Z,13E,17Z-eicosapentaenoic acid (15-HEPE), 15-hydroperoxy-5Z,8Z,11Z,13E,17Z-eicosapentaenoic acid (15-HpEPE), 5-hydroxy-6E,8Z,11Z,14Z-eicosatetraenoic acid (5-HETE), 8-hydroxy-5Z,9E,11Z,14Z-eicosatetraenoic acid (8-HETE), 9-hydroxy-5Z,7E,11Z,14Z-eicosatetraenoic acid (9-HETE), 12-hydroxy-5Z,8Z,10E,14Z-eicosatetraenoic acid (12-HETE), 15-hydroxy-5Z,8Z,11Z,13E-eicosatetraenoic acid (15-HETE), 5-hydroperoxy-6E,8Z,11Z,14Z-eicosatetraenoic acid (5-HpETE), 11-hydroperoxy-5Z,8Z,12E,14Z-eicosatetraenoic acid (11-HpETE), 12-hydroperoxy-5Z,8Z,10E,14Z-eicosatetraenoic acid (12-HpETE), 15-hydroperoxy-5Z,8Z,11Z,13E-arachidonic acid (15-HpETE), and mixtures thereof.

34. The method according to claim 30, wherein the at least one oxylipin is chosen from 10-TriHOME, 13-HODE, 9-HODE, 13-OxoODE, 9-OxoODE, 12,13-DiHOME, 15-HEPE and mixtures thereof.

35. The method according to claim 30, wherein the at least one oxylipin is present in an oleaginous plant extract enriched in oxylipins.

36. The method according to claim 30, wherein the oleaginous plant extract enriched in oxylipins is an extract of apple seeds, soybeans, hazelnuts, or gametophytes of brown algae.

37. The method according to claim 36, wherein the brown algae is Saccharina latissima enriched in oxylipins.
